(19) United States
(12) Patent Application Publication
(10) Pub. No.: US 2025/0275805 A1
(43) Pub. Date: Sep. 4, 2025

(54) RADIOFREQUENCY ABLATION ELECTRODE, USE THEREOF AND RADIOFREQUENCY ABLATION SYSTEM

(71) Applicant: MIANYANG LIDE ELECTRONICS CO., LTD., Mianyang (CN)

(72) Inventors: Fu MA, Mianyang (CN); Yongsheng LI, Mianyang (CN); Jue JIANG, Mianyang (CN); LI YANG, Mianyang (CN); Zhanchao ZHANG, Mianyang (CN); Yong ZHAO, Mianyang (CN)

(73) Assignee: MIANYANG LIDE ELECTRONICS CO., LTD., Mianyang (CN)

(21) Appl. No.: 18/268,939

(22) PCT Filed: Dec. 1, 2022
(86) PCT No.: PCT/CN2022/135833

§ 371 Date: Dec. 3, 2024

(30) Foreign Application Priority Data
Oct. 31, 2022 (CN) 202211350381.7

(51) Int. Cl. A61B 18/14; A61B 18/00

(52) U.S. Cl. CPC A61B 18/1477; A61B 2018/00023; A61B 2018/00065; A61B 2018/00577; A61B 2018/1425

(57) ABSTRACT

A radiofrequency ablation electrode is disclosed, and relates to the technical field of medical devices. The radiofrequency ablation electrode mainly includes an internal circulation structure and an external perfusion structure; the internal circulation structure enables a cooling medium in a liquid supply device to reach a working terminal of the radiofrequency ablation electrode, so as to cool the working terminal of the radiofrequency ablation electrode and a lesion tissue around the working terminal, and enables the cooling medium to flow back to the liquid supply device; and the external perfusion structure enables the cooling medium flow through micropores on the working terminal of the radiofrequency ablation electrode to reach the lesion tissue. A radiofrequency ablation system comprising the radiofrequency ablation electrode and a use of the radiofrequency ablation electrode in the preparation of medical devices are disclosed. According to the present disclosure, the electrical conductivity of the lesion tissue around the working terminal can be increased, the working impedance can be reduced, the cooling range can be expanded, and the carbonization of the lesion tissue can be prevented, thereby expanding the ablation range.

### TECHNICAL FIELD

The present disclosure relates to the technical field of medical devices, and in particular to a radiofrequency ablation electrode, a use thereof and a radiofrequency ablation system.

### BACKGROUND

Ablation is a minimally invasive operation, comprising chemical ablation and physical ablation. Physical ablation is commonly used in clinic, and includes radiofrequency ablation, microwave ablation, cryoablation, ultrasonic ablation, laser ablation and so on. Radiofrequency ablation is a mature and commonly used ablation method, which is mainly used to treat diseases such as nodules, blockages and tumors of human tissues and organs. Radiofrequency ablation treatment is to puncture an ablation electrode to a lesion site, release radiofrequency energy, increase the cell temperature of the lesion site, denature the lesion site, and finally make the tissue of the lesion site necrotic. The tissue can be absorbed and removed through normal metabolism of the human body, so as to achieve the purpose of eliminating nodules, unblocking blockages and eliminating tumors. Radiofrequency ablation treatment needs cooperate with a radiofrequency ablation system, including a radiofrequency ablation instrument and a radiofrequency ablation electrode.

With the popularization of physical examination, the number of cases with pulmonary nodules has gradually increased. On the contrary, the methods of treating pulmonary nodules are still very limited, and there are many shortcomings. At present, the main methods to treat pulmonary nodules comprise surgical resection, radiofrequency ablation and microwave ablation, in which the surgical resection has great trauma and high cost, and it is impossible to perform treatment for many times.

Compared with surgical resection, microwave ablation has less trauma and lower cost, but microwave needle has the following disadvantages.
(1) The microwave needle used in microwave ablation is usually a ceramic needle tip or a needle tip plated with Teflon on the copper surface. The needle tip is generally blunt, and cannot puncture through the skin, and needs to puncture with a skin-breaking needle. During lung puncture, the pulmonary nodules are hard, and the normal lung tissue is soft. The needle tip is blunt, and cannot puncture directly and accurately to the center of the lesion, and needs to puncture repeatedly in a positioning manner, which is easy to result in pulmonary hemorrhage.
(2) Before pulmonary nodule ablation, it is usually necessary to take biopsy in a positioning manner. In order to reduce the puncture time and puncture times, in clinic, the lesion tissue is usually first punctured through a coaxial needle. A biopsy gun is punctured into the lesion along a coaxial needle cannula to take biopsy, then an ablation needle is directly punctured into the lesion tissue along the coaxial needle cannula for ablation, and finally the needle channel is ablated, which can avoid the needle channel implantation of the biopsy channel and the risk of needle channel bleeding. Therefore, the ablation needle is required to be thinner so as to pass through the coaxial needle cannula. The coaxial needle cannula should not be too thick, which is easy to result in pneumothorax. Therefore, a thinner ablation needle is required. However, a microwave needle has a larger diameter, which is not suitable for pulmonary nodule ablation.
(3) The principle of microwave ablation determines that an antenna at a transmitting terminal will generate heat. Especially after the characteristic impedance near the working terminal changes, the microwave transmitting terminal will generate a large amount of heat, so that a ceramic needle sleeved on the transmitting antenna breaks and falls off, and the needle tip may be broken when working.

However, radiofrequency ablation needs to form a current loop. Most of the lung tissues are alveoli, and the electrical conductivity is not high. When using a single needle for radiofrequency ablation, a conventional cold circulation radiofrequency ablation needle only has an internal circulation cooling system. That is, the cooling medium reaches the external needle tubing from the liquid storage tank, and then returns to the liquid storage tank through the internal circulation cooling system, but no cooling medium enters the lesion tissue. The working terminal of the radiofrequency ablation needle is mostly alveoli, resulting in a small actual contact area between the working terminal and the lung tissue and a high initial working impedance. The initial impedance of tissues identified by a radiofrequency ablation host impedance identification system is high, which leads to no or small output power of the host. Even if there is output, a high voltage is required to ensure the output. Moreover, because there are few lung tissues actually in contact with the working terminal, a small part of lung tissues in contact with the working terminal may produce rapid carbonization, energy cannot be transferred, and a vicious circle is formed. The lesions are not ablated or the ablation range is not large in the image.

A conventional radiofrequency ablation perfusion single needle is only provided with a perfusion system. That is, the liquid medium reaches the front end of the outer needle tubing from the liquid storage tank and enters the lesion, which increases the electrical conductivity of the tissue. No liquid medium returns to the liquid storage tank, and the working terminal cannot be effectively cooled, resulting in partial blockage of the liquid injection holes on the working terminal due to blood coagulation and tissue carbonization during ablation. No liquid flows out of the blocked liquid injection holes, resulting in sharp carbonization. The ablation range is small. The flow rate of the unblocked liquid injection holes increases, so that the liquid medium is sprayed. The ablation shape is irregular, and it is easy to ablate the surrounding normal tissues.

At present, in order to reduce the working impedance of the radiofrequency ablation electrode, it is usually preferable to increase the contact area between the ablation electrode and the lung. In clinic, claw needles are often used to increase the contact area between the ablation electrode and the lung. Specifically, as shown in FIGS. 1 to 2, the claw needle includes a sub-needle 101, an inner needle tubing 102, an outer needle tubing 103, an insulating layer 104 and a needle tip 105. A plurality of sub-needles 101 are welded at the front end of the inner needle tubing 102, and the outer needle tubing 103 sleeves the inner needle tubing 102. The front end of the outer needle tubing 103 is the needle tip 105. The needle tip 105 has a beveled opening with a cutting edge. The outer needle tubing 103 is covered with the insulating layer 104. The needle tip 105 is exposed for skin and tissue puncture before ablation and needle channel ablation after ablation. During puncture, the sub-needle 101 is located in the outer needle tubing 103. After puncturing the lesion, the sub-needle 101 is pushed out of the outer needle tubing 103. The sub-needle 101 is deployed in the lesion, and then ablation is performed.

Although a claw needle can increase the contact area between the ablation electrode and the lung to a certain extent, the claw needle has the following disadvantages.
(1) The outer needle tubing needs to accommodate a plurality of sub-needles, which leads to the larger diameter of the outer needle tubing. However, the needle tip of the outer needle tubing is the outlet of the sub-needle, which is hollow and often has a beveled incision. Therefore, the needle tip is not sharp. The external needle tubing has a large diameter and is not sharp, so that which it is difficult to puncture pulmonary nodules.
(2) Because there are many sub-needles, it is impossible to see every sub-needle at the same time in the image, and there is a risk that a sub-needle will damage normal tissues.
(3) When the sub-needles are deployed in the pulmonary nodules, because the pulmonary nodules are hard and have large resistance, which may lead to the uneven deployment of two or more sub-needles. During ablation, the parts in which the sub-needles are not deployed form a hollow state, resulting in incomplete ablation.
(4) The main needle and each sub-needle of the claw needle usually have no cold circulation function. It is easy to result in tissue adhesion after ablation, so that it is difficult to retract the sub-needle.

It can be seen that the current method of using a claw needle to increase the contact area between the ablation electrode and the lung and thus reduce the working impedance has the above disadvantages, and the clinical application effect is poor. Therefore, it is urgent to provide a new method to reduce the working impedance of the radiofrequency ablation electrode.

### SUMMARY

The purpose of the present disclosure is to provide a radiofrequency ablation electrode, a use thereof and a radiofrequency ablation system, so as to solve the problems existing in the prior art, increase the electrical conductivity of the lesion tissue around the working terminal, reduce the working impedance, expand the cooling range, and prevent the carbonization of the lesion tissue, thereby expanding the ablation range.

In order to achieve the above purpose, the present disclosure provides the following scheme.

The present disclosure provides a radiofrequency ablation electrode, including an internal circulation structure and an external perfusion structure;
wherein the internal circulation structure enables a cooling medium in a liquid supply device to reach a working terminal of the radiofrequency ablation electrode, so as to cool the working terminal of the radiofrequency ablation electrode and a lesion tissue around the working terminal, and enables the cooling medium to flow back to the liquid supply device; and
the external perfusion structure enables the cooling medium to flow through micropores on the working terminal of the radiofrequency ablation electrode to reach the lesion tissue.

In some embodiments, the internal circulation structure includes an inner needle tubing and an outer needle tubing, the internal circulation structure includes an inner needle tubing and an outer needle tubing, the outer needle tubing sleeves the inner needle tubing and forms a fluid channel with the inner needle tubing, the cooling medium is capable of being introduced into the fluid channel, an inner needle tubing flow channel into which the cooling medium is capable of being introduced is provided in the inner needle tubing, a front end of the inner needle tubing flow channel is in communication with a front end of the fluid channel, a rear end of the inner needle tubing flow channel and a rear end of the fluid channel are both connected with the liquid supply device; the outer needle tubing is provided with a needle tip at a front end thereof and an insulating layer at a rear end thereof, and the outer needle tubing is also electrically connected with a radiofrequency head;
the external perfusion structure includes the micropores, and the micropores are formed in an area of the working terminal of the external needle tubing;
wherein the cooling medium is conductive, and the cooling medium is capable of entering the fluid channel and then overflowing from the micropores.

In some embodiments, the radiofrequency ablation electrode further includes a liquid cavity, the liquid cavity is located at the rear end of the outer needle tubing and configured to accommodate the cooling medium, and the liquid cavity is in communication with the fluid channel and the inner needle tubing flow channel; and
the liquid supply device includes a cooling medium source and a cooling medium recovery device, the liquid cavity includes a water inlet cavity and a backwater cavity, the water inlet cavity is separated from the backwater cavity; the water inlet cavity is connected with the cooling medium source through a water inlet pipe, the backwater cavity is connected with the cooling medium recovery device through a backwater pipe, the cooling medium source is capable of providing the cooling medium, the cooling medium recovery device is capable of recovering the cooling medium; a rear end of the inner needle tubing extends into the water inlet cavity, so that the rear end of the inner needle tubing flow channel is in communication with the water inlet cavity, and the rear end of the fluid channel is in communication with the backwater cavity.

In some embodiments, the water inlet pipe and/or the backwater pipe are further provided with a water-volume adjusting device, the water-volume adjusting device is configured to adjust a water inflow or a backwater volume of the cooling medium, so as to adjust a perfusion volume of the cooling medium; wherein the cooling medium is sterile physiological saline or liquid medicine, the perfusion volume of the cooling medium is a volume of the cooling medium entering a human body within unit time, and the perfusion volume of the cooling medium is 0.1 ml to 2.0 ml per minute.

In some embodiments, the pore size of the micropore is 0.005 mm to 0.05 mm.

In some embodiments, the insulating layer is an insulating tube, an outer sleeve also sleeves the front end of the outer needle tubing, the outer sleeve and the insulating tube are sequentially arranged from front to back in an axial direction of the outer needle tubing to form a protective tube, an outer wall of the protective tube is flush with an outer edge of the needle tip; wherein an outer wall of a front end of the outer sleeve is flush with the outer edge of the needle tip, an outer wall of a rear end of the outer sleeve is flush with an outer wall of a front end of the insulating tube; the outer sleeve is configured to release radiofrequency energy, and contrast holes are further formed on the outer sleeve.

In some embodiments, a plurality of rings of the micropores are formed on the outer needle tubing in the axial direction, a plurality of rings of the contrast holes are formed on the outer sleeve tube, the micropores and the contrast holes are staggered from front to back in the axial direction of the outer needle tubing; and after flowing out of the micropores, the cooling medium is capable of entering a gap between the outer needle tubing and the outer sleeve tube and flowing out of the contrast holes.

In some embodiments, the outer sleeve is a stainless steel metal tube, the insulating tube is a polymer plastic tube, the wall thicknesses of the outer sleeve and the insulating tube are both 0.01 mm to 0.1 mm; the needle tip is a triangular needle tip with a cutting edge, and the needle tip is welded at the front end of the outer needle tubing.

In some embodiments, the pore size of the micropores is 0.05 mm to 0.5 mm, and the gap between the outer sleeve and the outer needle tubing is 0.01 mm to 0.05 mm.

In some embodiments, the diameter of the outer needle tubing is at least 1.0 mm.

The present disclosure further provides a radiofrequency ablation system, which includes a radiofrequency ablation instrument and the radiofrequency ablation electrode described above.

The present disclosure further provides a use of the radiofrequency ablation electrode in the preparation of medical devices.

Compared with the prior art, the present disclosure has the following beneficial technical effects.

The radiofrequency ablation electrode of the present disclosure includes an internal circulation structure and an external perfusion structure. The internal circulation structure enables a cooling medium in a liquid supply device to reach a working terminal of the radiofrequency ablation electrode, so as to cool the working terminal of the radiofrequency ablation electrode and a lesion tissue around the working terminal, and enables the cooling medium to flow back to the liquid supply device. The cooling medium of the present disclosure can realize internal circulation, realize the cooling function of the radiofrequency ablation electrode, and simultaneously ensure that the micropores and the tiny gaps between the outer needle tubing and the outer sleeve are not blocked in the ablation process and no blood enters the tiny gaps and micropores so as to result in carbonization and adhesion, ensure that the cooling medium can continuously and uniformly overflow from each micropore, and ensure effective external perfusion of the cooling medium.

The external perfusion structure of the present disclosure enables the cooling medium flow through the micropores on the working terminal of the radiofrequency ablation electrode to reach the lesion tissue, and can cool the working terminal of the external needle tubing and the lesion tissue around the working terminal. The cooling medium can be injected into the lesion tissue, so that the electrical conductivity of the tissue is increased, the cooling range is expanded, the problems of carbonization and adhesion of the lesion tissue or blood in the energy injection process are effectively solved, the continuous input of energy is ensured, the ablation range is further expanded, and the ablation site is absorbed quickly after the patient is cured.

### BRIEF DESCRIPTION OF THE DRAWINGS

In order to explain the embodiments of the present invention or the technical scheme in the prior art more clearly, the drawings used in the embodiments will be briefly introduced hereinafter. Obviously, the drawings in the following description are only some embodiments of the present invention. For those skilled in the art, other drawings can be obtained according to these drawings without paying creative labor.

FIG. 1 is a schematic structural diagram of a claw needle in the prior art.

FIG. 2 is a cross-sectional view taken along line A-A in FIG. 1.

In FIG. 1 to FIG. 2, 101, sub-needle; 102, inner needle tubing; 103, outer needle tubing; 104, insulating layer; 105, needle tip.

FIG. 3 is a schematic structural diagram of a radiofrequency ablation electrode in Embodiment 1 of the present disclosure.

FIG. 4 is a schematic structural diagram of a radiofrequency ablation electrode in Embodiment 2 of the present disclosure.

FIG. 5 is a partial enlarged view of a working terminal part of a radiofrequency ablation electrode in Embodiment 2 of the present disclosure.

FIG. 6 is a schematic structural diagram of an outer needle tubing of a radiofrequency ablation electrode in Embodiment 2 of the present disclosure.

FIG. 7 is a schematic structural diagram of an outer sleeve of a radiofrequency ablation electrode in Embodiment 2 of the present disclosure.

FIG. 8 is a schematic assembly diagram of an outer sleeve and an outer needle tubing of a radiofrequency ablation electrode in Embodiment 2 of the present disclosure.

FIG. 9 is a schematic flow diagram of a cooling medium in an embodiment of the present disclosure.

FIG. 10 is a schematic diagram of perfusion of a cooling medium in Embodiment 2 of the present disclosure.

FIG. 11 is a schematic diagram of a coaxial needle in an embodiment of the present disclosure.

FIG. 12 is a cross-sectional view taken along line B-B in FIG. 11.

FIG. 13 is a cross-sectional view taken along line C-C in FIG. 11.

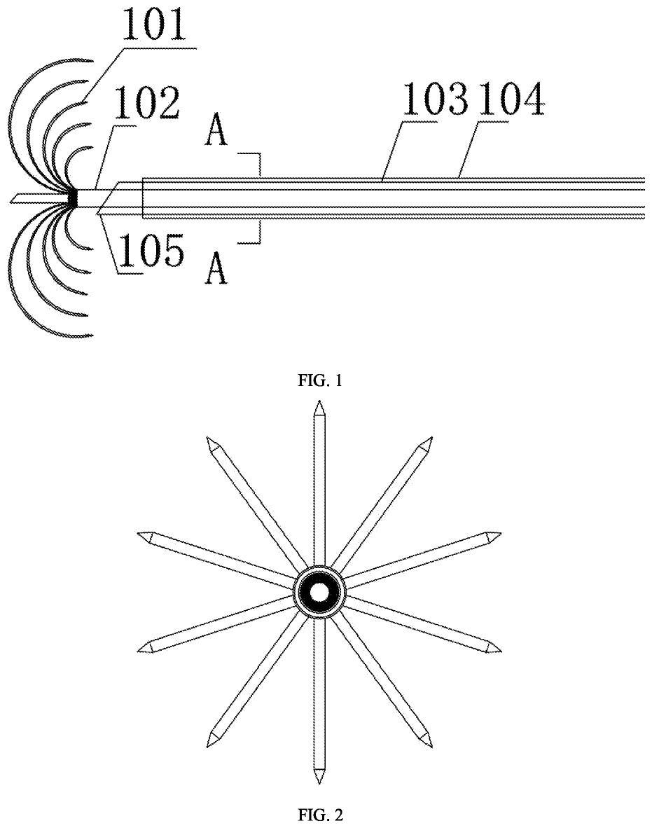

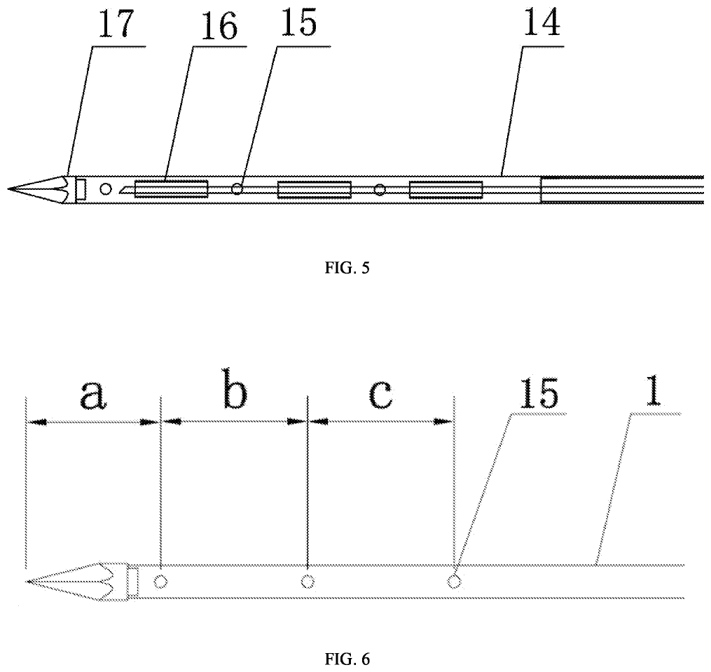

In FIG. 3 to FIG. 13, 1, outer needle tubing; 2, soldered dot; 3, handle; 4, water inlet cavity; 5, inner needle tubing; 6, backwater cavity; 7, inner conductor; 8, radiofrequency wire; 9, radiofrequency head; 10, water inlet pipe; 11, multi-gear adjusting switch; 12, backwater pipe; 13, insulating tube; 14, outer sleeve; 15, micropore; 16, contrast hole; 17, needle tip; 18, coaxial needle mandrel; and 19, coaxial needle cannula.

### DETAILED DESCRIPTION OF THE EMBODIMENTS

The technical scheme in the embodiments of the present disclosure will be described clearly and completely with reference to the drawings in the embodiments of the present disclosure. Obviously, the described embodiments are only some embodiments of the present disclosure, rather than all the embodiments. Based on the embodiments of the present disclosure, all other embodiments obtained by those skilled in the art without paying creative labor belong to the scope of protection of the present disclosure.

The purpose of the present disclosure is to provide a radiofrequency ablation electrode, a use thereof and a radiofrequency ablation system, so as to solve the problems existing in the prior art, increase the electrical conductivity of the lesion tissue around the working terminal, reduce the working impedance, expand the cooling range, and prevent the carbonization of the lesion tissue, thereby expanding the ablation range.

In order to make the above objects, features and advantages of the present disclosure more obvious and understandable, the present disclosure will be further explained in detail hereinafter with reference to the drawings and specific embodiments.

### Embodiment 1

As shown in FIG. 3, this embodiment provides a radiofrequency ablation electrode, which mainly comprises an internal circulation structure and an external perfusion structure; the internal circulation structure enables a cooling medium in a liquid supply device to reach a working terminal of the radiofrequency ablation electrode, so as to cool the working terminal of the radiofrequency ablation electrode and a lesion tissue around the working terminal, and enables the cooling medium to flow back to the liquid supply device; and the external perfusion structure enables the cooling medium flow through micropores on the working terminal of the radiofrequency ablation electrode to reach the lesion tissue.

In this embodiment, the internal circulation structure mainly comprises an inner needle tubing 5 and an outer needle tubing 1. The outer needle tubing 1 sleeves the inner needle tubing 5 and forms a fluid channel with the inner needle tubing 5. The cooling medium is capable of being introduced into the fluid channel. An inner needle tubing flow channel into which the cooling medium is capable of being introduced is provided in the inner needle tubing 5. The front end of the inner needle tubing flow channel is in communication with the front end of the fluid channel. The rear end of the inner needle tubing flow channel and the rear end of the fluid channel are both connected with the liquid supply device. The front end of the outer needle tubing 1 is provided with a needle tip 17, and the rear end of the outer needle tubing 1 is provided with an insulating layer. The insulating layer can insulate and protect the part of the outer needle tubing 1 that requires no energy release, and prevent the non-therapeutic part through which the radiofrequency ablation electrode passes from being thermally damaged. Further, the outer needle tubing 1 is a conductive metal tube. The outer needle tubing 1 can also be electrically connected with a radiofrequency head 9 through an inner conductor 7 and a radiofrequency wire 8, and sends current energy to the outer needle tubing 1 through the radiofrequency head 9. Specifically, the inner conductor 7 is welded to the rear end of the outer needle tubing 1 through a soldered dot 2. The inner conductor 7 is electrically connected with the radiofrequency head 9 through the radiofrequency wire 8. The radiofrequency head 9 is connected with a radiofrequency host to output radiofrequency energy.

The external perfusion structure mainly comprises the micropores 15, and the micropores 15 are formed in the working terminal area of the external needle tubing 1.

In this embodiment, the cooling medium is conductive, and the cooling medium is capable of entering the fluid channel and then overflowing from the micropores 15. The pore size of the micropore 15 is small, which can ensure that only a small amount of the cooling medium overflows from the micropores 15. The cooling medium overflows from the micropores 15, oscillates at the same frequency as the working frequency, and then forms high-temperature steam under the action of high temperature. The high-temperature steam only spread to the vicinity of the lesion tissue, increasing the electrical conductivity of the lesion tissue without flowing into other parts of the human body, thus reducing the damage to the human body as much as possible.

In the radiofrequency ablation electrode in this embodiment, the outer needle tubing 1 sleeves the inner needle tubing 5. A fluid channel is formed between the outer needle tubing 1 and the inner needle tubing 5. The working terminal area of the outer needle tubing 1 is provided with micropores 15. The conductive cooling medium can overflow through the micropores 15. The overflowed cooling medium increases the electrical conductivity of the lung tissue around the working terminal of the radiofrequency ablation electrode and reduces the working impedance. When radiofrequency ablation is performed, the lung tissue around the working terminal is thermally damaged due to heat accumulation. After thermal damage, alveoli in the lung tissue near the working terminal of the electrode needle are squeezed out, the lung lesion tissue contracts and collapses, and the contracted and collapsed lung tissue is closely combined and wrapped on the surface of the working terminal of the radiofrequency ablation electrode. Thereby, the contact area between the working terminal of the radiofrequency ablation electrode and the lung tissue is increased, which further reduces the working impedance, forms a low-impedance working environment, expands the ablation range, and overcomes the problems that a conventional radiofrequency single-needle has a high impedance during ablation and the radiofrequency energy of the host is not output or is output to a small scale.

Moreover, the cooling medium circulates in the fluid channel, which can cool the working terminal of the external needle tubing 1 and the lesion tissue around the working terminal. The cooling medium can be injected into the lesion tissue, the cooling range is expanded, the problems of carbonization and adhesion of the lesion tissue or blood in the energy injection process are effectively solved, the continuous input of energy is ensured, the ablation range is further expanded, and the ablation site is absorbed quickly after the patient is cured.

In this embodiment, the radiofrequency ablation electrode further comprises a liquid cavity, the liquid cavity is located at the rear end of the outer needle tubing and configured to accommodate the cooling medium, and the liquid cavity is in communication with the fluid channel and the inner needle tubing flow channel. Specifically, the liquid supply device comprises a cooling medium source and a cooling medium recovery device. The liquid cavity comprises a water inlet cavity 4 and a backwater cavity 6. The water inlet cavity 4 is separated from the backwater cavity 6. The water inlet cavity 4 is not in communication with the backwater cavity 6. The water inlet cavity 4 is capable of being connected with the cooling medium source through a water inlet pipe 10. The backwater cavity 6 is capable of being connected with the cooling medium recovery device through a backwater pipe 12. The cooling medium source is capable of providing the cooling medium. The cooling medium recovery device is capable of recovering the cooling medium. The rear end of the inner needle tubing 5 extends into the water inlet cavity 4, so that the rear end of the inner needle tubing flow channel is in communication with the water inlet cavity 4, the front end of the inner needle tubing flow channel is in communication with the front end of the fluid channel, and the rear end of the fluid channel is in communication with the backwater cavity 6.

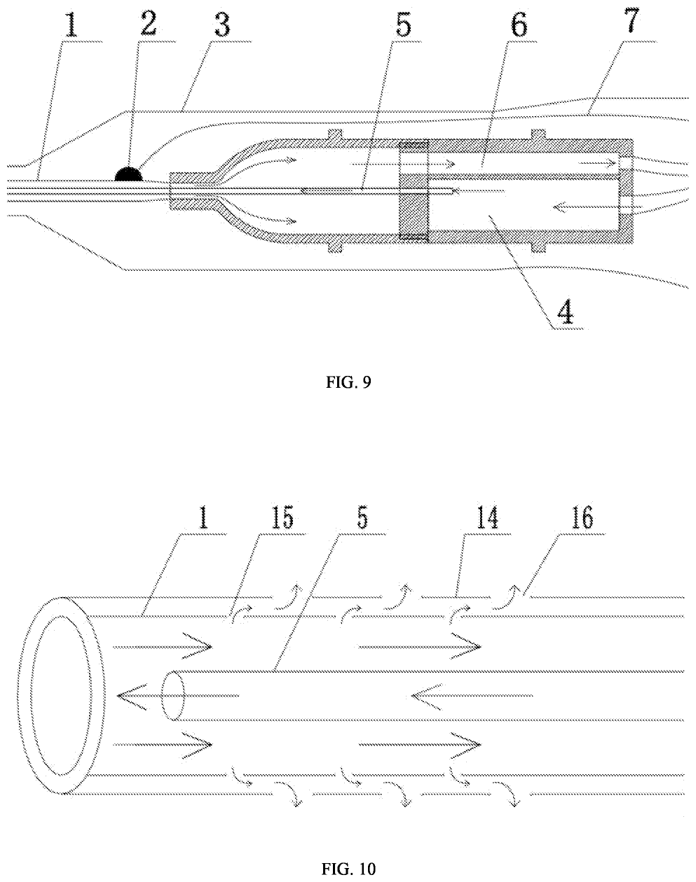

In this embodiment, as shown in FIG. 9, the cooling medium in the cooling medium source enters into the water inlet cavity 4 in the liquid cavity through the water inlet pipe 10, enters into the inner needle tubing flow channel through the rear end of the inner needle tubing 5, then enters into the front end of the fluid channel between the inner needle tubing 5 and the outer needle tubing 1 through the front end of the fluid channel, and finally flows into the cooling medium recovery device through the rear end of the fluid channel, the backwater cavity 6 and the backwater pipe 12 for recovery. During the flow of the cooling medium, the working terminal of the radiofrequency ablation electrode and the lesion tissue around the working terminal can be effectively cooled, and a small amount of the cooling medium can overflow from the micropores 15, which can increase the electrical conductivity of the lung tissue and effectively transmit the radiofrequency current energy.

In this embodiment, in order to realize the circulation of the cooling medium, the cooling medium source can be in communication with the cooling medium recovery device. Alternatively, the cooling medium source and the cooling medium recovery device are integrated, such as a liquid bottle. As a preferred embodiment, the cooling medium source and the cooling medium recovery device are integrated in this embodiment. Further, in order to make the cooling medium circulate smoothly, a circulating pump is provided between the liquid bottle and the water inlet pipe 10 to provide power for the circulation of the cooling medium. In this embodiment, the radiofrequency ablation electrode realizes the cold circulation function, which can ensure that the micropores 15 are not blocked during the ablation process, and no blood enters the micropores 15 so as to result in carbonization and adhesion, and ensure that the cooling medium can continuously and uniformly overflow from each micropore 15.

In this embodiment, the cooling medium is sterile physiological saline or liquid medicine, preferably cooled sterile physiological saline. The sterile physiological saline can increase the electrical conductivity of the tissue and effectively transmit radiofrequency current energy. After the sterile physiological saline is cooled, the temperature of the tissue near the working terminal can be reduced. Moreover, increasing the electrical conductivity of the tissue and reducing the temperature of the tissue can both expand the ablation range.

In this embodiment, the water inlet pipe 10 and/or the backwater pipe 12 are further provided with a water-volume adjusting device. The water-volume adjusting device is configured to adjust the water inflow or backwater volume of the cooling medium, so as to adjust the perfusion volume of the cooling medium. The water-volume adjusting device can be selected according to the specific working needs, such as a multi-gear adjusting switch 11 or a multi-gear hose buckle. As a preferred embodiment, in this embodiment, only the backwater pipe 12 is provided with the water-volume adjusting device. The perfusion volume of the cooling medium is controlled by controlling the return water. When the perfusion volume of the cooling medium is insufficient, the return water is reduced, and the perfusion volume is increased. When the perfusion volume is large, the return water is increased, and the perfusion volume is reduced.

Further, the perfusion volume of the cooling medium is preferably 0.1 ml to 2.0 ml per minute, wherein the perfusion volume of the cooling medium is the volume of the cooling medium entering the human body in unit time. In this embodiment, tiny-volume perfusion is used, and the perfusion volume of the cooling medium can be adjusted by the multi-gear adjusting switch 11. In the working process, a small amount of the perfused cooling medium oscillates at the same frequency as the working frequency to form high-temperature steam, which will not result in undesirable healing due to a large amount of liquid injected into the human body.

In this embodiment, the needle tip 17 is a triangular needle tip with a cutting edge, and the needle tip 17 is welded at the front end of the outer needle tubing 1. The needle tip 17 has high sharpness and can easily puncture skin and hard skin or tissues such as pulmonary nodules. The outer diameter of the outer needle tubing 1 can be at least 1.0 mm, which overcomes the pneumothorax problem resulted from the thick needle tubing during lung puncture.

In this embodiment, since the cooling medium flows directly into the human body after flowing out of the micropores 15, in order to ensure that only a small amount of the cooling medium flows into the human body, the pore size of the micropore 15 can be small, preferably 0.005 mm to 0.05 mm.

In this embodiment, in order to facilitate the operator to hold the radiofrequency ablation electrode, shield and protect the circuit and pipeline of the electrode during operation, a handle 3 is provided at the rear end of the outer needle tubing 1 and can wrap around the liquid cavity, and the tail of the handle 3 has an arc structure, which is ergonomic and convenient for the operator to hold, and the handle does not slip and is effortless when holding for a long time.

### Embodiment 2

This embodiment provides a radiofrequency ablation electrode, which is an improvement on the basis of Embodiment 1. Compared with Embodiment 1, the improvement in this embodiment is mainly shown as follows.

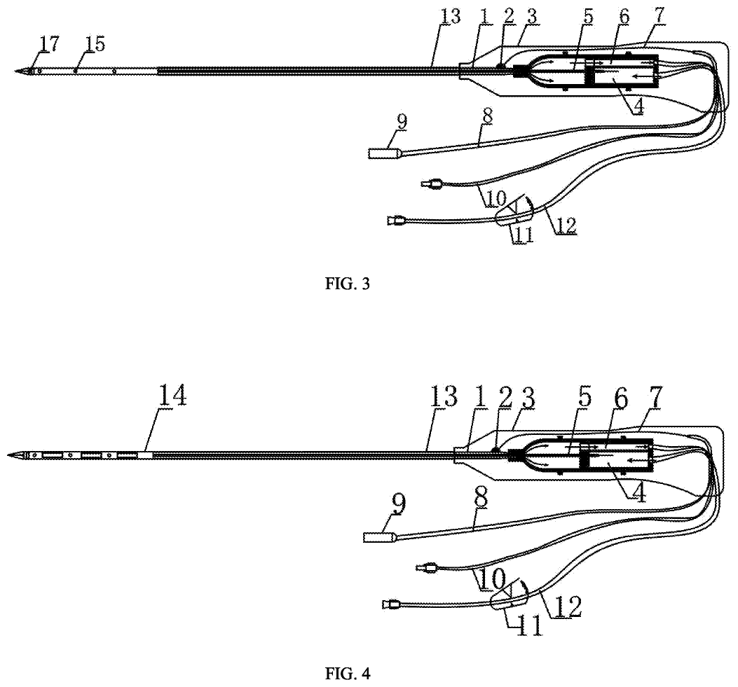

In this embodiment, as shown in FIGS. 4-8, the insulating layer is an insulating tube 13, which sleeves the rear end of the outer needle tubing 1. An outer sleeve 14 also sleeves the front end of the outer needle tubing 1. The outer sleeve 14 and the insulating tube 13 are sequentially arranged from front to back in the axial direction of the outer needle tubing 1 to form a protective tube. The outer wall of the protective tube is flush with the outer edge of the needle tip 17; wherein the outer sleeve 14 is used to release radiofrequency energy, and contrast holes 16 are further formed on the outer sleeve 14. The insulating tube 13 can insulate and protect the part of the outer needle tubing 1 that requires no energy release, and prevent the non-therapeutic part through which the radiofrequency ablation electrode passes from being thermally damaged.

In this embodiment, the inner needle tubing 5, the outer needle tubing 1 and the protective tube are all preferably round tubes, or square tubes or other polygonal prism tubes can be selected as required.

In this embodiment, a plurality of rings of the micropores 15 are formed on the outer needle tubing 1 in the axial direction, a plurality of rings of the contrast holes 16 are formed on the outer sleeve tube 14 in the axial direction, and the micropores 15 and the contrast holes 16 are staggered from front to back in the axial direction of the outer needle tubing 1. That is, in the axial direction, the position of the micropores 15 on the outer needle tubing 1 corresponds to the tube wall of the outer sleeve 14 (the position where the contrast holes 16 are not formed). The micropores 15 can be shielded by the tube wall of the outer sleeve 14, so as to prevent the cooling medium from being sprayed. However, as shown in FIG. 10, there is a tiny gap between the outer sleeve 14 and the outer needle tubing 1. After flowing out of the micropores 15, the cooling medium is capable of entering the gap between the outer needle tubing 1 and the outer sleeve tube 14 and flowing out of the contrast holes 16. In order to ensure the tiny-volume perfusion of the cooling medium, the gap between the outer sleeve 14 and the outer needle tubing 1 is preferably 0.01 mm to 0.05 mm.

The cold circulation function of the radiofrequency ablation electrode in this embodiment also ensures that the micropores 15 and the tiny gap between the outer needle tubing 1 and the outer sleeve 14 are not blocked during the ablation process, and no blood enters the tiny gap and the micropores 15 so as to result in carbonization and adhesion, and ensures that the cooling medium can continuously and uniformly overflow from each micropore.

Further, after flowing out of the micropores 15, the cooling medium can overflow into the human body through the gap between the outer needle tubing 1 and the outer sleeve tube 14 and the contrast holes 16. In this embodiment, the micropore 15 can have a larger pore size than Embodiment 1. Tiny-volume perfusion can also be realized under the larger pore size. The pore size of the micropore 15 is preferably 0.05 mm to 0.5 mm. The processing difficulty of the outer needle tubing 1 can be reduced to some extent by providing the micropore 15 with a larger pore size.

Moreover, the outer sleeve 14 is provided with contrast holes 16, and an uneven surface of the working terminal can be formed, which creates a developing function through an imaging device, thus solving the problem that the working terminal develops an image not clearly under the imaging device, especially avoiding the problem that each sub-needle of the claw needle cannot be seen through the imaging device at the same time in the prior art. In this embodiment, accurate puncture can be realized in the puncture process by judging the position of the working terminal, so as to prevent erroneous puncture resulted from the fact that the working terminal cannot develop an image.

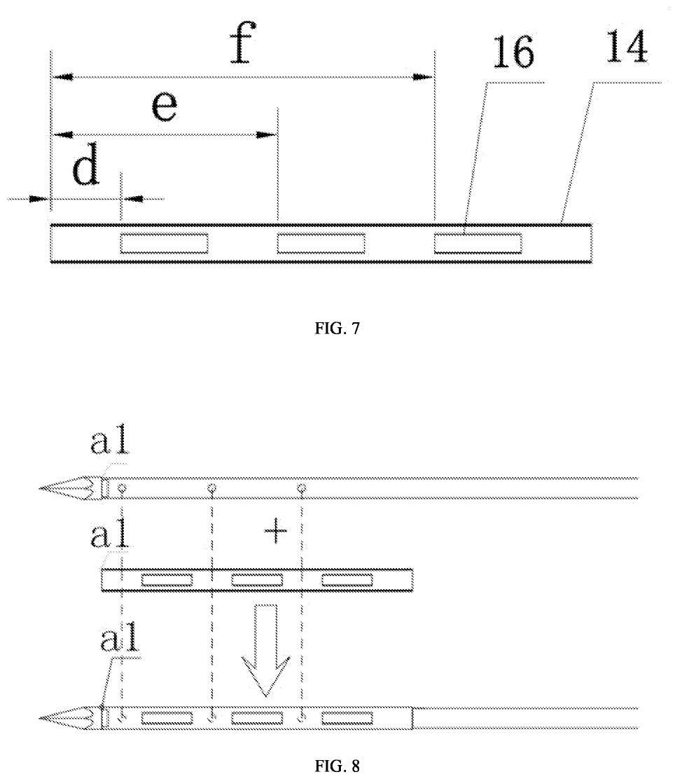

As a preferred embodiment, in this embodiment, as shown in FIGS. 6-7, three rings of micropores 15 are provided. The three rings of micropores 15 are provided on three circumferences on the outer needle tubing 1 at distances a, a+b and a+b+c, respectively, from the tip of the front end of the needle tip 17. The centers of the micropores 15 are located on the corresponding circumferences, there are three micropores 15 in each ring, the three micropores 15 are uniformly distributed on the circumference, and thus there are nine micropores 15 in total. The three micropores 15 in each ring are corresponding to three micropores 15 in other ring. The connecting lines between the centers of the corresponding micropores 15 are parallel to the axis of the outer needle tubing 1. Further, three rings of contrast holes 16 are correspondingly provided. The three rings of contrast holes 16 are provided on three circumferences at distances d, e and f, respectively, from the front end of the outer sleeve 14. The front ends of the contrast holes 16 are located on the corresponding circumferences, and there are three contrast holes 16 in each ring, the three contrast holes 16 are uniformly distributed on the circumference, and thus there are nine contrast holes 16 in total. The three contrast holes 16 in each ring are corresponding to three contrast holes 16 in other ring. The connecting lines between the centers of the corresponding contrast holes 16 are parallel to the axis of the outer sleeve 14. Three micropores 15 in each ring correspond to three contrast holes 16 in each ring respectively. Moreover, the plane where the center connecting line of the micropores 15 and the contrast holes 16 corresponding to the micropores 15 are located is parallel to the axis of the outer needle tubing 1.

In this embodiment, a is preferably 4.5 mm to 8.5 mm, b is preferably 5 mm to 9 mm, c is preferably 5.5 mm to 9.5 mm, d is preferably 2.25 mm to 4.25 mm, e is preferably 8.5 mm to 12.5 mm, and f is preferably 15.75 mm to 19.75 mm. The distance between each micropore 15 and the tip of the front end of the needle tip 17 and the distance between each contrast hole 16 and the front end of the outer sleeve 14 can be selected according to the working needs, specifically, according to the length of the working terminal of the radiofrequency ablation electrode.

In this embodiment, the number of rings of the micropores 15 and the contrast holes 16 and the number of the micropores and the contrast holes in each ring can be selected as required. For example, four or five rings can be provided, and each ring can be provided with four or five holes. The number of rings of the micropores 15 and the contrast holes 16 and the number of the micropores and the contrast holes in each ring may be the same or different. Further, the shapes of the micropores 15 and the contrast holes 16 can also be selected according to the specific working needs, such as square holes or round holes. As a preferred embodiment, the micropores 15 are round holes, and the contrast holes 16 are square holes.

In this embodiment, the fluid channel between the inner needle tubing 5 and the outer needle tubing 1 can be an annular fluid channel, and the annular gap between the inner needle tubing 5 and the outer needle tubing 1 is the fluid channel. Alternatively, the fluid channel is an axial channel, and a plurality of axial channels are uniformly distributed along the circumference between the inner needle tubing 5 and the outer needle tubing 1. The axial channels correspond to the micropores 15 on each ring one by one. As a preferred embodiment, the fluid channel in this embodiment is an annular fluid channel.

In this embodiment, the outer sleeve 14 and the insulating tube 13 can be integrally provided or separately provided, preferably separately provided. The outer sleeve 14 sleeves the front end of the outer needle tubing 1, and the insulating tube 13 sleeves the part of the outer needle tubing 1 that requires no energy release. The outer wall of the front end of the outer sleeve 14 is flush with the outer edge of the needle tip 17, and the outer wall of the rear end thereof is flush with the outer wall of the insulating tube 13. During puncture, the front end of the insulating tube 13 can be prevented from squeezing and wrinkling with the skin tissue, resulting in lengthening of the exposed working terminal and damaging normal tissues.

In this embodiment, the outer sleeve 14 is preferably a thin-walled stainless steel metal tube, the front end of which is welded to the rear end of the needle tip 17. The welding point al between the outer sleeve 14 and the needle tip 17 and the welding point al between the outer needle tubing 1 and the needle tip 17 coincide. The front end of the outer sleeve 14 and the front end of the outer needle tubing 1 are welded. The communication between the outer needle tubing 1 and the outer sleeve 14 can be realized during connection, so that the outer sleeve 14 can be used for radiofrequency energy release. The insulating tube 13 is preferably a thin-walled insulating polymer plastic tube. The wall thicknesses of the insulating tube 13 and the outer sleeve 14 are both preferably 0.01 mm to 0.1 mm. The insulating tube 13 is preferably made of Teflon, or PEEK, polyimide and other materials as required.

In this embodiment, the outer sleeve 14 can also be made of insulating material. At this time, radiofrequency energy can be released through the contrast hole 16 on the outer sleeve 14. The front end of the outer sleeve 14 can be connected with the needle tip 17 by clamping or bonding.

### Embodiment 3

This embodiment provides a radiofrequency ablation system, comprising a radiofrequency ablation instrument and a radiofrequency ablation electrode in Embodiment 1 or Embodiment 2.

### Embodiment 4

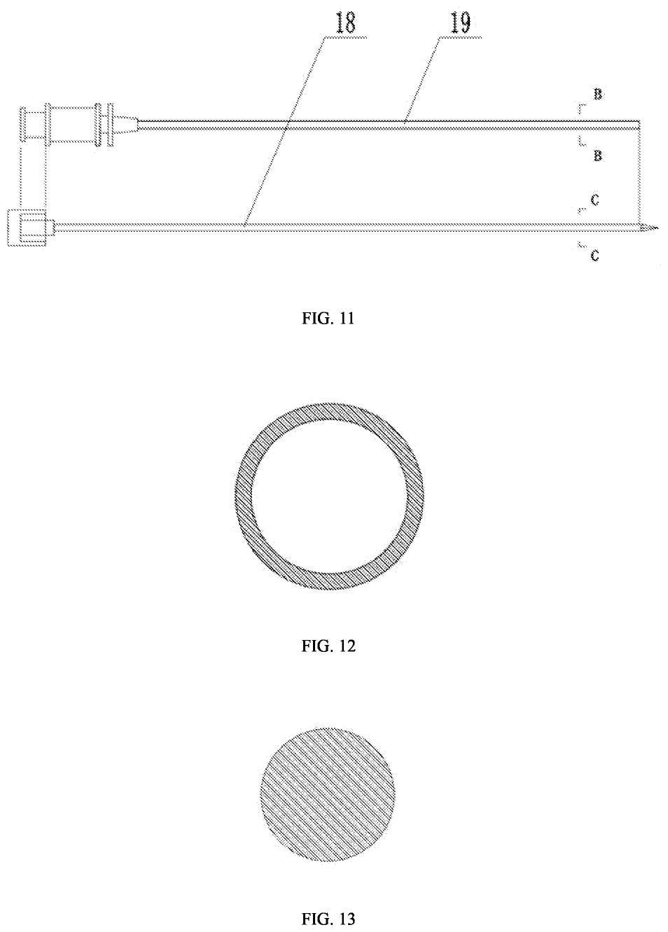

This embodiment provides a use of the radiofrequency ablation electrode in Embodiment 1 or Embodiment 2 in the preparation of medical devices. Specifically, in this embodiment, the radiofrequency ablation electrode can be prepared into a biopsy ablation device together with a coaxial needle and a biopsy gun. As shown in FIG. 11 to FIG. 13, a coaxial needle cannula 19 is matched with a coaxial needle mandrel 18. During the operation, the coaxial needle mandrel 18 is first inserted into the coaxial needle cannula 19 to puncture the skin to reach the lesion. After puncturing to the lesion, the coaxial needle mandrel 18 is pulled out, and the biopsy gun is inserted into the lesion through the coaxial needle cannula 19 to take a biopsy. Then the biopsy gun is pulled out. The radiofrequency ablation electrode is inserted through the coaxial needle cannula 19 to ablate the lesion. After completing ablation, the coaxial needle cannula 19 is pulled out together with the radiofrequency ablation electrode, and the radiofrequency ablation electrode is used to ablate the needle channel. It should be noted that during radiofrequency ablation, the working terminal of the radiofrequency ablation electrode penetrates through the coaxial needle cannula 19. The distance between the front end of the insulating tube 13 on the outer needle cannula 1 and the front end of the coaxial needle cannula 19 is more than 1 cm, so that the insulating tube cannot be in contact with the coaxial needle cannula, otherwise the coaxial needle cannula 19 will be conductive.

In this embodiment, the outer diameter of the outer needle tubing 1 can be at least 1.0 mm. The overall diameter of the radiofrequency ablation electrode is small, so that the radiofrequency ablation electrode can be inserted into the coaxial needle cannula 19 for radiofrequency ablation, which solves the problem in the prior art that the microwave needle or the claw needle cannot penetrate into the coaxial needle due to the thick needle tubing, completes biopsy and ablation in the same puncture channel, avoids secondary puncture, ablates the puncture channel after ablation, and avoids bleeding after multiple punctures and needle channel implantation.

In this embodiment, the radiofrequency ablation electrode can also be used in conjunction with other medical devices except the biopsy gun according to specific working needs.

Based on the basic principle of radiofrequency ablation (radiofrequency ablation aims to cause the thermal damage of the tissue through the principle of resistance thermal effect and heat conduction, and then solidify for necrosis, so as to achieve the purpose of ablating nodules, tumors, etc.), the electrical conductivity of the lesion tissue is increased by perfusing a small amount of the cooling medium, and the lesion temperature is reduced by cold circulation to prevent tissue carbonization. The lesion impedance can be reduced, which is beneficial to the continuous output of radiofrequency energy and expands the range of radiofrequency ablation.

Moreover, besides the cold circulation function and the liquid perfusion function, the present disclosure also has the function of adjusting perfusion flow. The present disclosure is small in diameter and clear in development under the imaging device, which is a safe and effective radiofrequency ablation electrode which can be applied to diseases such as lung tumors and pulmonary nodules. It should be further noted that the present disclosure includes but is not limited to the treatment of lung tumors, nodules and other diseases. According to the principle and structure of the present disclosure, the radiofrequency ablation electrode for other lesions and other diseases can be understood as being within the scope of protection of the present disclosure.

It should be noted that it is obvious to those skilled in the art that the present disclosure is not limited to the details of the above exemplary embodiments, and can be realized in other specific forms without departing from the spirit or basic features of the present disclosure. Therefore, no matter from any point of view, the embodiments should be regarded as exemplary and non-restrictive, and the scope of the present disclosure is defined by the appended claims rather than the above description. Therefore, it is intended to encompass all changes that fall within the meaning and scope of equivalent elements of the claims within the present disclosure, and any reference signs in the claims should not be regarded as limiting the claims involved.

In the present disclosure, a specific example is applied to illustrate the principle and implementation of the present disclosure, and the explanation of the above embodiments is only used to help understand the method and its core idea of the present disclosure. At the same time, according to the idea of the present disclosure, there will be some changes in the specific implementation and application scope for those skilled in the art. To sum up, the contents of this specification should not be construed as limiting the present disclosure.

What is claimed is:

1. A radiofrequency ablation electrode, comprising an internal circulation structure and an external perfusion structure;
wherein the internal circulation structure enables a cooling medium in a liquid supply device to reach a working terminal of the radiofrequency ablation electrode, so as to cool the working terminal of the radiofrequency ablation electrode and a lesion tissue around the working terminal, and enables the cooling medium to flow back to the liquid supply device; and
the external perfusion structure enables the cooling medium to flow through micropores on the working terminal of the radiofrequency ablation electrode to reach the lesion tissue.

2. The radiofrequency ablation electrode according to claim 1, wherein:
the internal circulation structure comprises an inner needle tubing and an outer needle tubing, the outer needle tubing sleeves the inner needle tubing and forms a fluid channel with the inner needle tubing, the cooling medium is capable of being introduced into the fluid channel, an inner needle tubing flow channel into which the cooling medium is capable of being introduced is provided in the inner needle tubing, a front end of the inner needle tubing flow channel is in communication with a front end of the fluid channel, a rear end of the inner needle tubing flow channel and a rear end of the fluid channel are both connected with the liquid supply device; the outer needle tubing is provided with a needle tip at a front end thereof and an insulating layer at a rear end thereof, and the outer needle tubing is also electrically connected with a radiofrequency head;
the external perfusion structure comprises the micropores, and the micropores are formed in an area of the working terminal of the external needle tubing; and
wherein the cooling medium is conductive, and the cooling medium is capable of entering the fluid channel and then overflowing from the micropores.

3. The radiofrequency ablation electrode according to claim 2, wherein the radiofrequency ablation electrode further comprises a liquid cavity, the liquid cavity is located at the rear end of the outer needle tubing and configured to accommodate the cooling medium, and the liquid cavity is in communication with the fluid channel and the inner needle tubing flow channel; and
the liquid supply device comprises a cooling medium source and a cooling medium recovery device, the liquid cavity comprises a water inlet cavity and a backwater cavity, the water inlet cavity is separated from the backwater cavity; the water inlet cavity is connected with the cooling medium source through a water inlet pipe, the backwater cavity is connected with the cooling medium recovery device through a backwater pipe, the cooling medium source is capable of providing the cooling medium, the cooling medium recovery device is capable of recovering the cooling medium; a rear end of the inner needle tubing extends into the water inlet cavity, so that the rear end of the inner needle tubing flow channel is in communication with the water inlet cavity, and the rear end of the fluid channel is in communication with the backwater cavity.

4. The radiofrequency ablation electrode according to claim 3, wherein the water inlet pipe and/or the backwater pipe are further provided with a water-volume adjusting device, the water-volume adjusting device is configured to adjust a water inflow or a backwater volume of the cooling medium, so as to adjust a perfusion volume of the cooling medium; wherein the cooling medium is sterile physiological saline or liquid medicine, the perfusion volume of the cooling medium is a volume of the cooling medium entering a human body within unit time, and the perfusion volume of the cooling medium is 0.1 ml to 2.0 ml per minute.

5. The radiofrequency ablation electrode according to claim 2, wherein a pore size of each micropore is 0.005 mm to 0.05 mm.

6. The radiofrequency ablation electrode according to claim 2, wherein the insulating layer is an insulating tube, an outer sleeve also sleeves the front end of the outer needle tubing, the outer sleeve and the insulating tube are sequentially arranged from front to back in an axial direction of the outer needle tubing to form a protective tube, an outer wall of the protective tube is flush with an outer edge of the needle tip; wherein an outer wall of a front end of the outer sleeve is flush with the outer edge of the needle tip, an outer wall of a rear end of the outer sleeve is flush with an outer wall of a front end of the insulating tube; the outer sleeve is configured to release radiofrequency energy, and contrast holes are further formed on the outer sleeve.

7. The radiofrequency ablation electrode according to claim 6, wherein a plurality of rings of the micropores are formed on the outer needle tubing in the axial direction, a plurality of rings of the contrast holes are formed on the outer sleeve tube, the micropores and the contrast holes are staggered from front to back in the axial direction of the outer needle tubing; and after flowing out of the micropores, the cooling medium is capable of entering a gap between the outer needle tubing and the outer sleeve tube and flowing out of the contrast holes.

8. The radiofrequency ablation electrode according to claim 6, wherein the outer sleeve is a stainless steel metal tube, the insulating tube is a polymer plastic tube, wall thicknesses of the outer sleeve and the insulating tube are both 0.01 mm to 0.1 mm; the needle tip is a triangular needle tip with a cutting edge, the needle tip is welded at the front end of the outer needle tubing; a pore size of each micropore is 0.05 mm to 0.5 mm, and a gap between the outer sleeve and the outer needle tubing is 0.01 mm to 0.05 mm.

9. The radiofrequency ablation electrode according to claim 2, wherein the diameter of the outer needle tubing is at least 1.0 mm.

10. A radiofrequency ablation system, comprising a radiofrequency ablation instrument and the radiofrequency ablation electrode according to any of claims 1 to 9.

11. Use of the radiofrequency ablation electrode according to any one of claims 1 to 9 in the preparation of medical devices.
